(19) United States
(12) Patent Application Publication
(10) Pub. No.: US 2013/0217000 A1
(43) Pub. Date: Aug. 22, 2013

(54) METHOD FOR CHARACTERISING A BIOLOGICALLY ACTIVE BIOCHEMICAL ELEMENT BY ANALYSING LOW FREQUENCY ELECTROMAGNETIC SIGNALS

(71) Applicant: Luc Montagnier, Le Plessis-Robinson (FR)

(72) Inventor: Luc Montagnier, Le Plessis-Robinson (FR)

(21) Appl. No.: 13/846,123

(22) Filed: Mar. 18, 2013

Related U.S. Application Data
(63) Continuation of application No. 12/097,204, filed on Oct. 16, 2008.

(30) Foreign Application Priority Data
Dec. 14, 2005 (FR) FR05/12686

(51) Int. Cl. G01N 33/487; C12Q 1/02

(52) U.S. Cl. CPC G01N 33/48735; C12Q 1/02
USPC 435/5; 435/29; 435/287.1

(57) ABSTRACT

A method for characterizing a biologically active biochemical element in a sample by prefiltering the sample and analyzing low frequency electromagnetic signals transmitted by the prefiltered solution. The prefiltering may be through a 150 nm or less filter. The prefiltering may be subsequent to a dilution, e.g., between 10⁻² and 10⁻²⁰ in water. The filtered sample may be stirred and/or centrifuged. During the analyzing, the solution may be excited using white noise. The analyzing may comprise comparing a signature with previously recorded signatures.

This application is a continuation of U.S. application Ser. No. 12/097,204, filed Oct. 16, 2008, which was a National Stage application under 35 U.S.C. §371 of PCT/FR2006/002735, filed Dec. 14, 2006, and claims benefit of foreign priority under 35 U.S.C. §119 from FR05/12686, filed Dec. 12, 2005. The contents of the above mentioned applications are hereby incorporated by reference in their entireties.

The present invention relates to the field of the characterisation of biochemical material from microorganisms or the structural or molecular components thereof, through the analysis of the electromagnetic signals generated after a filtering, and preferably after a dilution stage.

From the studies by Professor Jacques Benveniste, it is known to store and digitalise the specific activity of a biologically active molecule. The molecules analysed in the prior art are a natural substance (histamine, caffeine, nicotine, adrenaline . . . ) or drugs.

In the prior art, it was provided to sense this signal and to transmit it in an analogical or preferably digital form.

Within the scope of such studies, the European patent EP0701695 discloses a method and a device for transmitting, in the form of a signal characterising the demonstration of the biological activity or the biological behaviour specific to a determined substance. It also discloses the processing of such a signal from a first carrier material having said biological activity to a second material physically separated from the first material, and initially free of any physical presence of said determined substance, and a material obtained through such a method. This method of the prior art includes the amplification of the electric or electromagnetic signal emitted by the first substance and sensed by a sensor, and the transmission to an emitter of a signal characterising the demonstration of the biological activity or the biological behaviour of the first material, then the detection in the second material of a signal characterising the demonstration of a biological activity specific to said determined substance and transmitted to such second material through high-gain amplification means.

The French patent FR2811591 is also known, which discloses a method for producing signals and more particularly, electric signals, characterising the biological and/or chemical activity of a studied substance, to process a receiving substance initially having no particular biological activity, more particularly water, so that it has a biological activity after being processed. The receiving substance after the processing is called hereinafter the “Processed Substance” (or Informed Material). When the receiving substance is water, the Processed Substance is called “Processed Water” (or Informed Water). The substance having a biological activity can also be in the form of a preparation or homeopathic granules.

The international patent application WO0001412 discloses a method for activating an inactive solution and having a very low concentration of a biological and/or chemical determined substance in a solvent, consisting in placing such solution in a mechanical excitation field and in submitting such solution to a stirring for creating such mechanical excitation field. The concentration of said determined substance in said solution is lower than 10⁻⁶ moles per liter.

The object of the present invention is to provide improvements to such technique in order to extend the field of application and the performances thereof.

For this purpose, the invention, in its broadest sense, relates to a method for characterising a biologically active biochemical element by analysing low frequency electromagnetic signals transmitted by a solution prepared from an analysable material sample, characterised in that it includes a pre-filtering stage.

Preferably, the sample is filtered through a filter having a porosity of less than or equal to 150 nanometers prior to the analysis stage and more particularly, a porosity between 20 nanometers and 100 nanometers.

Advantageously, the dilution stage consists of a dilution between 10⁻² and 10⁻²⁰ and more particularly, between 10⁻² and 10⁻⁹.

According to a preferred embodiment, the method includes a strong stirring stage and/or a centrifuging stage.

According to a preferred embodiment, the solution is excited by means of a white noise during the acquisition of the electromagnetic signals.

The invention more particularly relates to the application of the characterising method to the analysis of microorganisms.

It also relates to the biological analysis consisting in recording the signatures obtained through the application of the characterising method to known biochemical elements, and in comparing the signature obtained to that of a biochemical element to be characterised with the previously recorded signatures.

The invention also relates to a biological inhibition method consisting in recording at least one signature obtained through the application of the characterising method to at least one known biochemical element, and in applying an inhibition signal depending on said signature to a sample.

It also relates to an equipment for the biological analysis including a sensor for the acquisition of the electromagnetic signals transmitted by a solution through the implementation of the characterising method according to the invention, a circuit for processing said signals for calculating a signature of an analysed sample and a circuit for comparing the thus computed signature with a base of previously recorded signatures.

The invention will be better understood upon reading the following description and referring to the appended drawings which correspond to non-limitative exemplary embodiments, wherein:

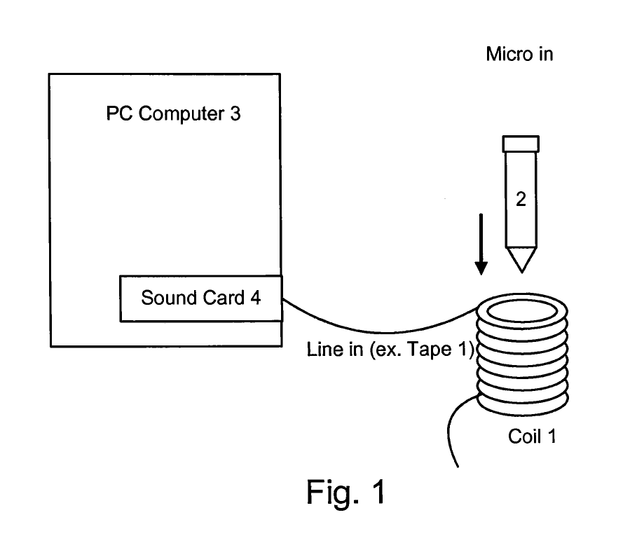

FIG. 1 shows a schematic view of the signal acquisition equipment;

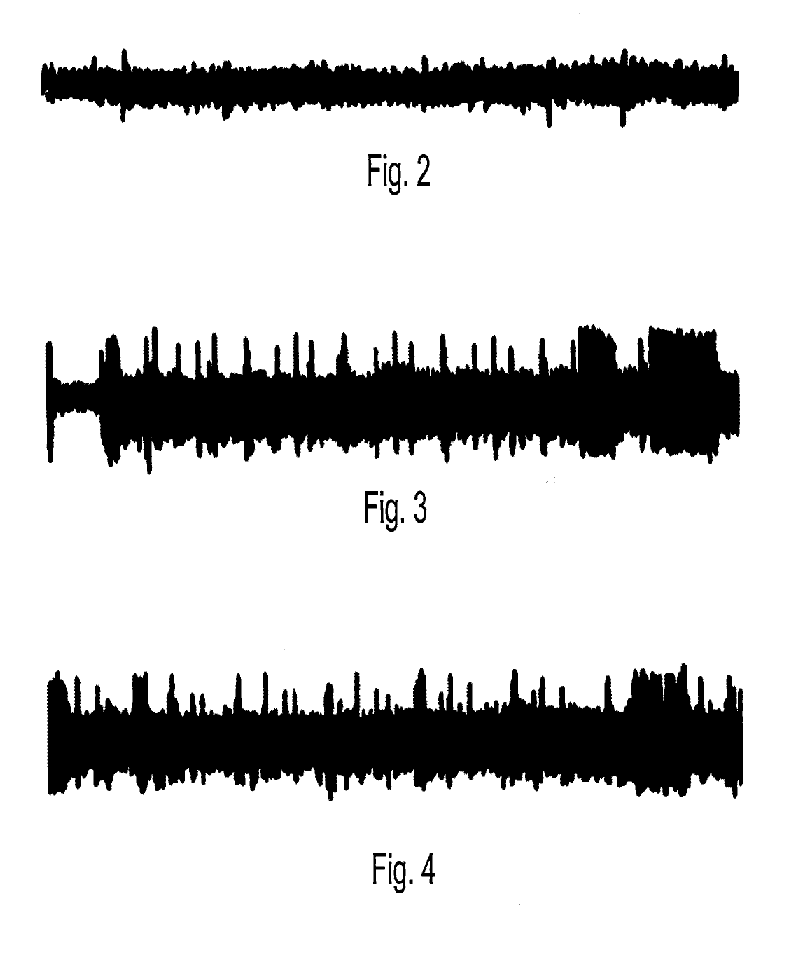

FIG. 2 shows a view of the electric signals generated by the solenoid in the absence of any emitting source (background noise);

FIGS. 3 and 4 show views of the electric signals generated by the solenoid in the presence of an emitting source (Mycoplasma pirum) after the filtering with 0.02 micrometer and 0.1 micrometer;

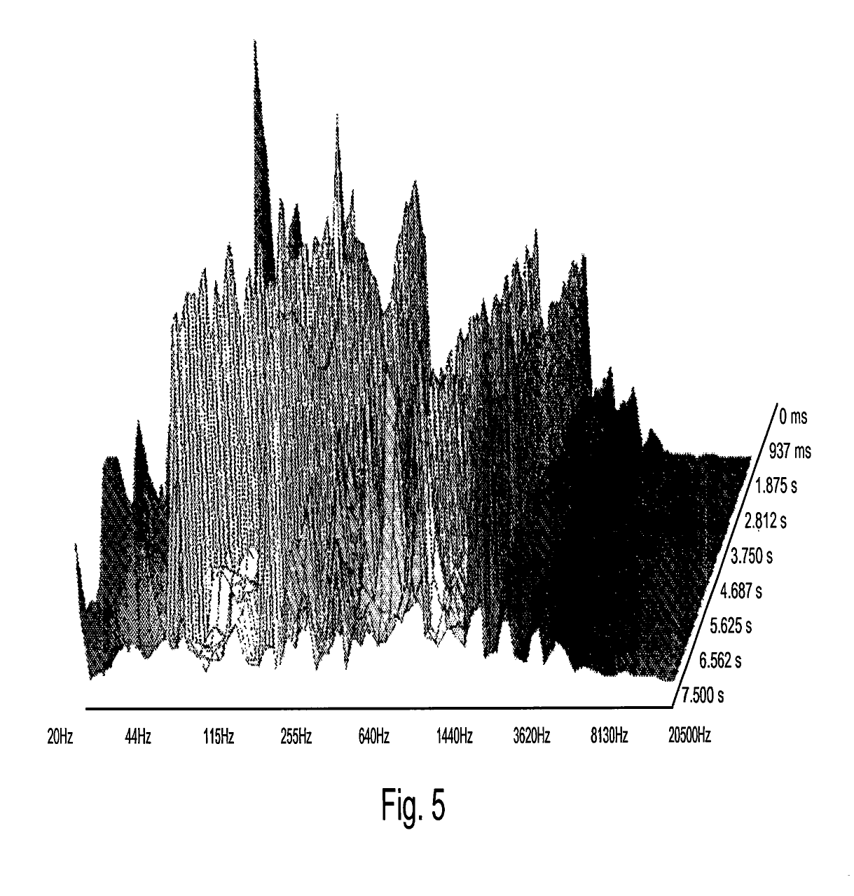

FIG. 5 shows a three-dimension amplitude histogram of the distribution of the wavelengths detected by the solenoid in the absence of any emitting source (background noise);

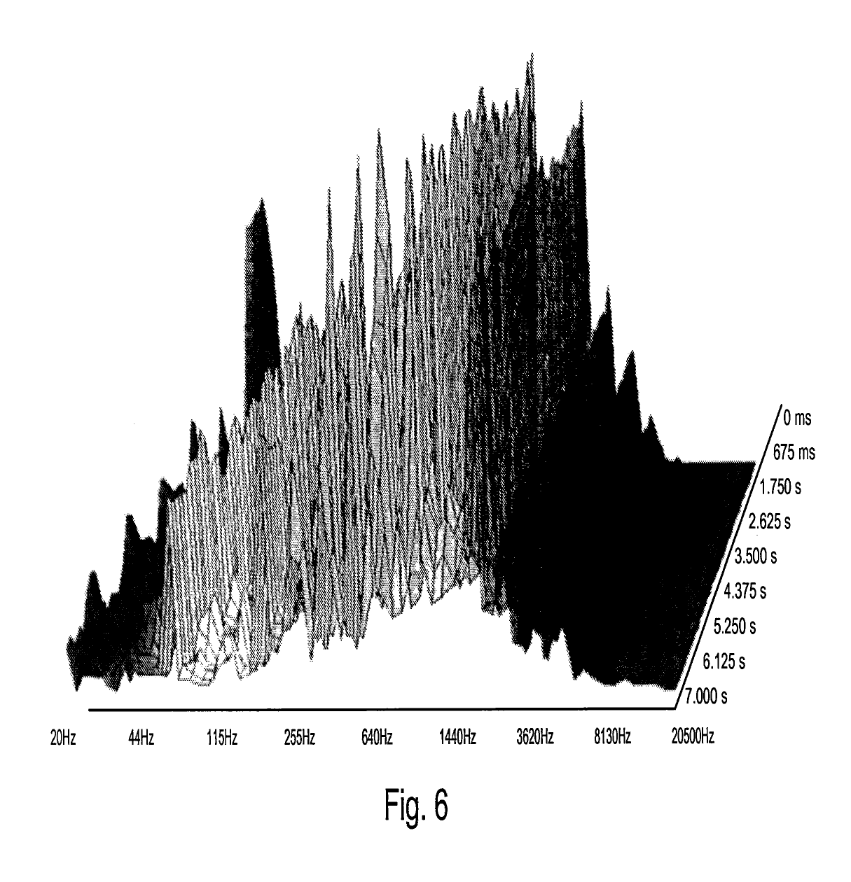

FIG. 6 shows a three-dimension amplitude histogram of the distribution of the wavelengths detected by the solenoid in the presence of an emitting source (Mycoplasma pirum) after a 0.02 micrometer filtering;

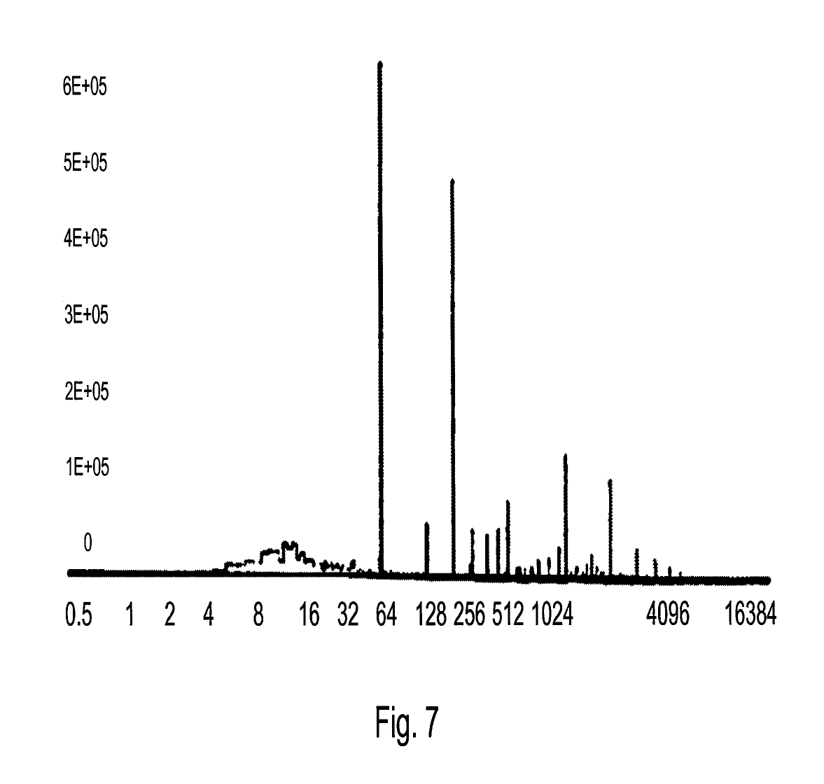

FIG. 7 shows a Fourier analysis of the same background noise as shown in FIG. 5 (non-filtered harmonics of the supply electric current);

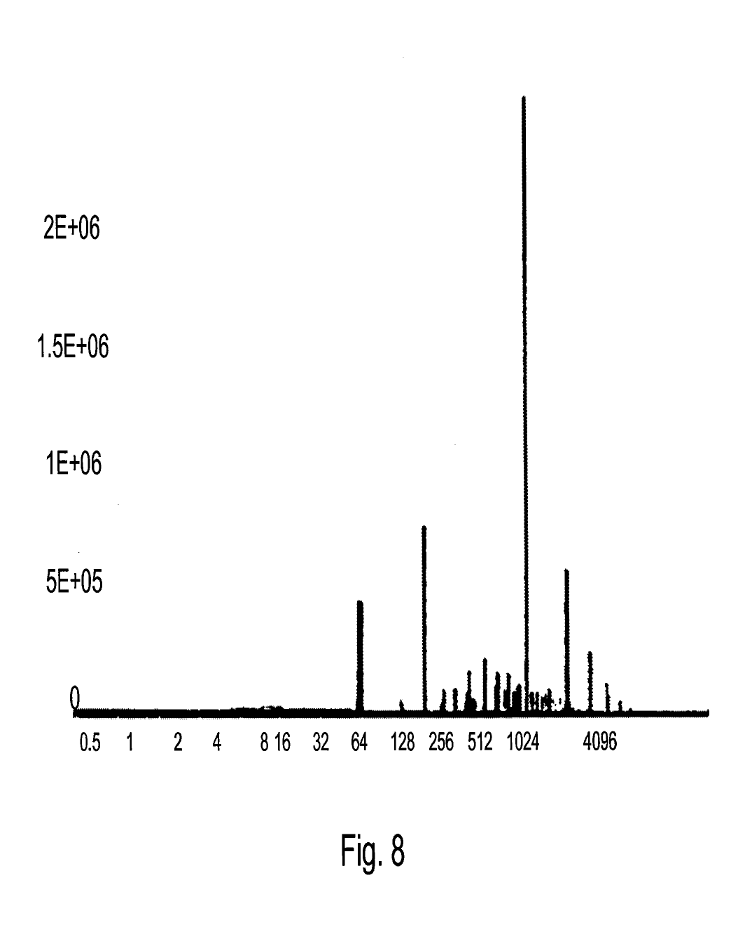

FIG. 8 shows a Fourier analysis of a signal generated by the solenoid in the presence of an emitting source as shown in FIG. 6 (Mycoplasma pirum);

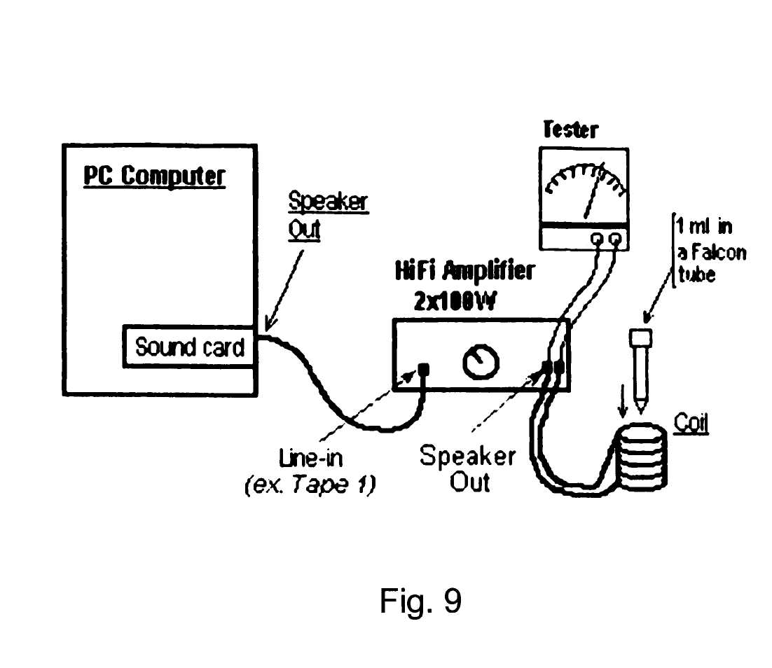

FIG. 9 shows a schematic view of the amplification device for the application of a previously recorded signal.

In the following, it shall be noted:
That the living organisms are suspended in the in vitro or in vivo culture medium in blood samples, more particularly a plasma sample from a person taking an anti-coagulant, preferably heparin.
That the nanostructures emitting signals are isolated from the filtered culture medium or plasma to eliminate any living organism (0.45 micrometer, then 0.1 micrometer or 0.02 micrometer for bacteria, 0.45 micrometer, then 0.02 micrometer for viruses).
That the electromagnetic signals are recorded on a computer and can be represented in different ways:
Globally, as measured on 6 seconds twice in a row, the signal being considered as positive when the magnitude thereof reaches at least 1.5 times that of the background noise
during an analysis in the form of a three-dimension histogram
during an analysis through the Fourier transform.

The present description discloses the implementation of an exemplary method according to the invention, for characterising three examples of microorganisms, through the analysis of emitted signals:
Mycoplasma Mycoplasma pirum (M. pirum)
HIV (Human Immuno Deficiency Virus), strain IIIB (LAI)
Bacterium Escherichia coli K12 (E. coli)
Plasma from HIV-infected patients.

### Experiment 1: Application to a Culture of M. Pirum in CEM Cells.

A culture of M. pirum in CEM cells is prepared in an rpmi 1640 culture medium+10% of foetal bovine serum. The cells in good condition show the presence of typical aggregates related to the presence of M. pirum.

The suspension is centrifuged at low speed for eliminating the cells. The supernatant fluid is filtered on a 0.45μ PEVD Millipore® (Merck KGAA, Darmstadt Germany) filter, then the filtrate is filtered again on a 0.02μ Whatman Anatop® (Whatman International Limited, Springfield Mill, Kent UK) filter, or a 0.1μ Millipore® filter.

Then a comparison is made with a supernatant fluid from not infected CEM cells, filtered under the same conditions. The solutions are 10 by 10 diluted in a complete rpmi under a laminar flow hood up to 10⁻⁷. Then each solution is processed in a Vortex (maximum power) for 15 seconds prior to the following dilution.

The detection of signals is performed with equipment shown in a schematic view in FIG. 1. The equipment includes a reading solenoid cell (1) with a sensitivity between 0 and 20,000 hertz, positioned on a table made of an isolating material. The solutions to be read are distributed in plastic (2) Eppendorf® (Eppendorf AG Barkhausenweg, Hamburg, Germany) conical tubes, 1.5 milliliters in capacity. The liquid volume is generally 1 milliliter, in a few cases 0.3 to 0.5 milliliter, without any difference in the answer to be noted. Each sample is read for 6 seconds, twice in a row, and each reading is entered separately.

The electric signals delivered by the solenoid are amplified using an audio card (4) up to a computer (3) the appropriate software of which gives a visual representation of the recorded elements:

An amplitude raw global representation is given in FIGS. 2, 3 and 4. Some background noise (−) can be noted (FIG. 2) and it is averaged. A positive signal is detected when the amplitude exceeds at least 1.5 times the background noise, defined as (+). In general, the detected amplitude is twice and sometimes three times, the background noise (++): the detected signal will be called a EMS (ElectroMagnetic Signal).
A 3D histogram analysis, respectively of the background noise and the signal in presence of the sample is shown in FIGS. 5 and 6.
A breakdown into individual frequencies through Fourier transform of the background noise and the signal respectively in the presence of the sample is shown in FIGS. 7 and 8.

### Results:

### 1) Emission of EMSs

Non-filtered suspension: a background noise (−) can be noted in the non-infected control and in the infected suspension. FIG. 2 is the amplitude raw global representation of the detected signal.

0.02 micrometer filtered solution. FIG. 3 is the amplitude raw global representation of the detected signal: a clear difference can be noted. The solution from the mycoplasma suspension is (++) up to the 10⁻⁷ dilution. The non-infected CEM control is (−). An additional experiment, performed a few hours later from the 10⁻⁶ dilution makes it possible to recover a positivity (++) up to the 10⁻¹⁴ and (+) up to the 10⁻¹⁵ dilutions. The 10⁻⁶ and the 10⁻⁷ dilutions in the first experiment remain (++) after several hours at 20° C.

0.1μ filtered solution. FIG. 4 is the amplitude raw global representation of the detected signal. The M. pirum filtrate is (++) until the 10⁻⁷ dilution. The controls are all negative except for 1 reading of the 10⁻² dilution. It should be noted that the 8 control tubes are close to the M. pirum tubes, positioned in the same plastic support. The positivity of one of the tubes can be explained by the passage of the signals from one tube to another, through their walls.

Fourier analysis of the positive frequencies shows in descending intensity order: 1,000, 2,000, 3,000, 1,999, 999, 2,999, 500, 399, 300, 900, for 10⁻⁶ and 10⁻⁷ (all using 0.02μ filtrate).

The 3D analysis (FIG. 6) shows a displacement of magnitude peaks towards the highest frequencies in the positive elements (+), as compared to the control (FIG. 5).

### Experiment 2: Behaviour of the EMS Source During Centrifugation at the Balance of Density, in Gradient, of 20 to 70% Saccharose in PBS

A centrifuging is carried out for 2 hours at 35,000 revolutions per minute at +4° C., starting from the first 0.02μ filtrate preserved overnight at +4° C. Its positivity is checked just prior to the centrifuging.

Upon completion of the centrifugation, 12 fractions are taken from the bottom of the tube. Measuring refraction indices makes it possible to determine the density gradient.

Fractions are then grouped 2 by 2 and diluted up to 10⁻⁷ in a rpmi 1640 medium+a 10% concentration bovine serum.

| | |
|---|---|
| Pool 1-2, density 1.26-1.28 | Pool 3-4, density 1.25-1.26 |

| | | |
|---|---|---|
| (−) for all the dilutions | Not diluted | (−) |
| | 10⁻¹ | (−) |
| | 10⁻² | (+) |
| | 10⁻¹ | (−) |
| | 10⁻⁴ | (++) |
| | 10⁻⁵ | (++) |
| | 10⁻⁶ | (++) |
| | 10⁻⁷ | (−) |

The negativity of the less diluted fractions can be explained by a self-interference of the signals emitted by too many sources. Such self-inhibition is checked by mixing 0.1 milliliter of the non-diluted element with 0.4 milliliter of the 10⁻⁴ dilution: after a vortex processing, a failing of the signal which does become negative can be efficiently noted.

| | |
|---|---|
| Pool 5-6, density 1.21-1.225 | Pool 7-8, density 1.165-1.194 |

| | | | |
|---|---|---|---|
| Not diluted | (−) | Not diluted | (−) |
| 10⁻¹ | (−) | 10⁻¹ | (−) |
| 10⁻² | (−) | 10⁻² | (+) |
| 10⁻¹ | (−) | 10⁻¹ | (−) |
| 10⁻⁴ | (−) | 10⁻⁴ | (−) |
| 10⁻⁵ | (++) | 10⁻⁵ | (++) |
| 10⁻⁶ | (++) | 10⁻⁶ | (++) |
| 10⁻⁷ | (+) | 10⁻⁷ | (++) |

| Pool 9-10, density 1.112-1.114 | Pool 11-12-13 (high) |
|---|---|
| Not diluted at 10⁻⁷ (−) | Not diluted at 10⁻⁷ (−) |

It can be noted that the source of the electromagnetic signals behaves like a polymer having a large size (but <0.02μ) and a density between 1.16 and 1.26.

A zone effect which had not been seen with the non-centrifuged raw preparation must also be mentioned. A self-interference occurs for the dilutions up to 10⁻¹ with a peak of activity (5-6 and 7-8).

### Experiment 3: Application to a Culture of HIV1/IIIB Infected CEM Cells.

Such experiment relates to HIV1/IIIB infected CEM cells culture prepared in two steps:
4 days: beginning of the cyto-pathogen effect (CPE)
6 days: CPE++effect

It is compared with a control culture of non-infected CEM.

The operating procedure includes the following steps:
0.45 micrometer filtering of the supernatant fluid
then 0.02 micrometer filtering
by 10 dilution of the filtrate up to 10⁻⁷ in a RPMI medium+bovine serum
strong stirring in a vortex for 15 seconds at each dilution step.

### Results:

1) with the 4-day culture, no signal above the background noise can be noted. There is no difference with non-infected CEM control up to the 10⁻⁷ dilution.

2) with the 6-day infected culture:
10⁻¹ to 10⁻⁶ (−)
10⁻⁶ (++)
10⁻⁷ (++)
10⁻⁸ (++)
10⁻⁹-10⁻¹⁵ (−)

A self-interference experiment is carried out again:

0.1 ml of the 10⁻¹ (negative) solution+0.4 ml of the 10⁻⁷ (positive) solution: the latter becomes negative. A self-interference does occur with low dilutions.

### Behaviour of the EMS Source During Centrifugation at the Balance of Density, in Gradient, of 20 to 70% Saccharose in PBS

3) Analysis in density gradient

The supernatant fluid of the positive culture filtered on a 0.02 micrometer filter is centrifuged at the balance of density in gradient, of 20-70% saccharose at 35,000 revolutions per minute in a BECKMAN° (Beckman Instruments, Inc. Fullerton Calif.) SW56 rotor at 4° C.

A control supernatant fluid of non-infected CEM cells is processed in a similar way

After centrifuging, 13 fractions are collected and grouped 2 by 2. The refraction indices of some fractions are determined with an Abbe refractometer in order to determine the density gradient.

The 400 ml fractions are diluted in a RPMI 1640 medium plus bovine serum. Successive dilutions are carried out 10 by 10 from such fractions.

It can be noted that the groups having a 1.23-1.24 and 1.19-1.21 density are very positive up to a 10⁻⁷ dilution. The 1.15-1.16 density group gives positive signals up to the 10⁻⁷ dilution. The group at the top of the tube gives no signal, whatever the dilution.

The fraction groups from the bottom of the tube (1.25 to 1.28 in density) give positive signals for a few dilutions only.

Contrary to M. pirum, self-interference occurs for the starting filtrate and no self-interference occurs from the gradient fractions.

Most signals in this case focus, as with M. pirum, in fractions having a 1.19 to 1.26 density, with a shoulder towards the lighter 1.16 fractions.

### Experiment 5: M. Pirum EMS Source Inactivation Test

One milliliter of a 10⁻¹ diluted 0.02 micrometer filtrate of M. Pirum is placed in an Eppendorf® tube. Such tube is placed in a solenoid supplied for 10 minutes with the previously recorded raw electric signal previously recorded on a M. Pirum preparation having the same dilution, after amplification.

FIG. 9 shows a schematic view of the equipment, including a computer 3 provided with a sound card (4) the outlet of which is connected to an amplifier (10) having a maximum power of 60 watts, in the example described. The amplified signal is applied to a flexible solenoid (11) in which the Eppendorf® tube (12) is placed. The signal applied is measured with a piece of equipment (13).

Various types of amplified signals are applied for 10 minutes to the M. Pirum suspension which gives a positive signal.

a) The same signal, but amplified: the starting signal remains positive. On the contrary, a control tube containing the 0.02 micrometer filtrate of non-infected CEM cells which was negative becomes positive. This suggests that the electromagnetic signals can be transmitted in a non-active medium provided that the initial spectrum has not been modified.

b) If the highest intensity frequencies (179, 374, 624, 1,000, 2,000 Hertz) are selected in the spectrum of the electromagnetic signals emitted by nanostructures of M. Pirum, the signal also remains positive, after the application of such amplified frequencies.

c) On the contrary, if the same signals with a phase inversion are applied, the EMS positivity disappears.

This is also true when all the EMS emitted by M. Pirum with a phase inversion are used.

d) It is also possible to neutralise the signals by allo-interference, i.e. signals from another microorganism (E. coli).

Experiment 4: Analysis of the Plasma from Persons Having Various Infections (HIV, Ureaplasma urolyticum Urethritis and Rheumatoid Arthritis).

Such analysis shows that such plasmas, once filtered and diluted in an appropriate way, transmit signals which are analogous to those transmitted by the same microorganisms, in vitro, except for the polyarthritis for which the infecting causes have not been identified yet.

More particularly, in the case of AIDS infected patients treated by anti-retrovirus tri-therapy, such signals are emitted by high dilutions of plasma (up to 10⁻¹⁶), which suggests that they exist abundantly after the disappearance of the plasmatic virus charge and could contribute in the residual infection remaining after the treatment.

### General Conclusion

Microorganisms of different nature, such as retrovirus (HIV), bacteria without rigid walls close to Gram+ (M. pirum), bacteria with rigid walls Gram− (E. coli) give nanostructures held in aqueous solutions.

After the indispensable step of filtering, which eliminates physical particles of microorganisms, such nanostructures (having a size of less than 100 nanometers) emit complex electromagnetic signals at low frequencies which can be recorded and digitised.

The same results can be obtained from the plasma of patients infected by such microorganisms.

Such nanostructures are different from the microorganisms which generated them by their large spectrum intensity and their sensitivity to deep-freezing. The signals they emit can be neutralised by self-interference with the previously recorded and phase reverse signals or through allo-interference with the signals from other microorganisms.
1 A method for characterising a biologically active biochemical element, by analysing low frequency electromagnetic signals transmitted by a solution prepared from an analysable material sample, characterised in that it comprises a pre-filtering stage.
2 A method for characterising a biochemical element according to 1, characterised in that, prior to the analysis stage, the sample is filtered through a filter having a porosity of less that 150 nanometres.
3 A method for characterising a biochemical element according to 2, characterised in that, prior to the analysis stage, the sample is filtered through a filter having a porosity between 20 nanometres and 100 nanometres.
4 A method for characterising a biochemical element according to 1, 2 or 3, characterised in that the dilution stage consists of a dilution between 10⁻² and 10⁻²⁰
5 A method for characterising a biochemical element according to 3, characterised in that the dilution 20 level is between 10⁻² and 10⁻⁹
6 A method for characterising a biochemical element according to 1, characterised in that it includes a strong stirring stage.
7 A method for characterising a biochemical element according to 1, characterised in that it includes a centrifuging stage.
8 A method for characterising a biochemical element according to anyone of the preceding 1-7, characterised in that the solution is excited using a white noise during the acquisition of the electromagnetic signals.
9—Application of the characterising method according to at least one of the preceding 1-8 for the analysis of microorganisms.
10 A method for characterising a biochemical element consisting in:
recording the signatures obtained through the analysis of the low frequency electromagnetic signals transmitted by a solution prepared from the known biological samples after a previous filtering stage, with a filter having a porosity of less than or equal to 150 nanometres, prior to the analysis stage, and more particularly a porosity between 20 nanometres and 100 nanometres,
recording the signatures obtained through the analysis of the low frequency electromagnetic signals transmitted by a solution prepared from the biological samples to be characterised after a previous filtering stage with a filter having a porosity of less than or equal to 150 nanometres prior to the analysis stage, and more particularly a porosity between 20 nanometres and 100 nanometres, and comparing the signature of the element to be characterised with the previously recorded signatures.
11. Application of the characterising method according to 1 to the biological inhibition, characterised in that it includes a stage of recording at least one signature of a biologically active biochemical element, consisting in analysing the low frequency electromagnetic signals transmitted by a solution prepared from an analysable material known from a previous filtering stage with a filter having a porosity of less than or equal to 150 nanometres, prior to the analysis stage, and in particular a porosity between 20 nanometres and 100 nanometres and after applying an inhibition signal depending on said signature to a sample.
12 Equipment for characterising a biochemical element according to the method of 1, said equipment including means for preparing a solution from a sample with a filter having a porosity of less than or equal to 150 nanometres prior to the analysis stage and in particular, a porosity between 20 nanometres and 100 nanometres, a sensor for acquiring the electromagnetic signals transmitted by a solution, a circuit for processing said signals for calculating a signature for an analysed sample and a comparison circuit for comparing the signature so computed with a base of previously recorded signatures.

1. A method for characterising a biochemical material sample comprising:
preparing a solution from the biochemical material sample;
pre-filtering the solution through a filter having a porosity of 150 nm or less and optionally diluting, centrifuging, agitating, and/or stirring it;
detecting a low frequency electromagnetic signal signature emitted by the prefiltered solution characteristic of the biochemical material sample; and optionally
recording said signature, displaying said signature, and/or comparing said signature with a signature obtained from another biochemical material sample.

2. The method of claim 1 wherein said preparing comprises isolating a living organism in an in vivo or in vitro culture medium.

3. The method of claim 1, wherein said preparing comprises isolating a living organism in a plasma sample.

4. The method of claim 1, wherein said preparing comprises removing living organism from the sample.

5. The method of claim 1, wherein said preparing comprises removing a living organism that is HIV (human immunodeficiency virus), Ureaplasma urolyticum urethritis or rheumatoid arthritis from a plasma sample.

6. The method according to claim 1, wherein said prefiltering comprises filtering the solution through a filter having a porosity of less than 150 nanometers and agitating it prior to said detection.

7. The method according to claim 1, wherein said prefiltering comprises filtering the solution through a filter having a porosity of between 20 nanometers and 100 nanometers and agitating it prior to said detection.

8. The method according to claim 1 that comprises diluting the biochemical material sample by between 10⁻² and 10⁻¹⁶ to form the solution.

9. The method according to claim 1 that comprises diluting the biochemical material sample by between 10⁻² and 10⁻⁹.

10. The method according to claim 1 that comprises stirring the solution prior to detecting a low frequency electromagnetic signal signature emitted by the prefiltered solution.

11. The method according to claim 1 that comprises centrifuging the solution prior to detecting a low frequency electromagnetic signal signature emitted by the prefiltered solution

12. The method according to claim 1 that comprises exciting the solution using a white noise excitation signal prior to detecting a low frequency electromagnetic signal signature emitted by the prefiltered solution.

13. The method according to claim 1, wherein said detecting comprises acquiring signals of less than 20,000 Hz.

14. The method of claim 1 that comprises recording said signature.

15. The method of claim 1 that comprises recording at least one signature of a solution formed from a biochemical material, wherein said filtering is performed through a filter having a porosity of less than or equal to 150 nanometers and after applying an inhibition signal selectively dependent on said at least one signature to a sample.

16. The method of claim 1 that comprises characterizing a biochemical element by:
automatically recording a set of signatures obtained through a predetermined analysis of low frequency electromagnetic signals transmitted by a solution prepared from identified biological samples after a previous filtering stage using an automated analyzer, with a filter having a porosity of less than or equal to 150 nanometers;
automatically recording at least one signature obtained through the predetermined analysis of the low frequency electromagnetic signals transmitted by a solution prepared from a biological sample to be characterized after a previous filtering stage using the automated analyzer, with a filter having a porosity of less than or equal to 150 nanometers, and
comparing the at least one signature with the recorded set of signatures.

17. The method of claim 1 comprising:
filtering of a solution prepared from a diluted sample of biological material through a filter having a pore size less than about 150 nm;
mechanically stirring the filtered solution;
acquiring low frequency electromagnetic signals less than about 20 kHz over time from the filtered solution;
analyzing the acquired low frequency electromagnetic signals, by performing at least one frequency domain transformation using an automated processor to produce at least one representation of the acquired low frequency electromagnetic signals which selectively varies in dependence on an organism present in the biological material; and
producing at least one output in dependence on said analyzing.

18. The method of claim 1 comprising characterizing a biological activity by:
storing at least one signature obtained through automated analysis of electromagnetic signals of low frequencies emitted by a solution prepared from an identified biological sample after a preliminary filtration step, with a filter having a porosity of less than or equal to about 150 nanometers, prior to the respective analysis;
obtaining at least one signature through automated analysis of the electromagnetic signals of low frequencies emitted by a solution prepared from a biological sample to be characterized, after a preliminary filtration step, with a filter having a porosity of less than or equal to about 150 nanometers, prior to the respective analysis; and
characterizing the at least one signature of the unknown biological sample by comparing it with at least one stored signature of the identified biological sample.

19. The method of claim 1 comprising characterizing a biological activity by:
performing a preliminary stage of filtration of a solution prepared from a sample of biological material; and
performing an automated analysis of electromagnetic signals of low frequencies emitted by the filtered solution using an automated analyzer, to produce an output dependent on a characteristic of a biological activity of the sample of biological material.

20. The method of claim 1 that comprises comparing signatures acquired from different biochemical material samples.

21. The method of claim 1 that comprises comparing signatures acquired from samples obtained from different microorganisms.

22. Equipment for characterising a biochemical element according to the method of claim 1, said equipment including means for preparing a solution from a sample with a filter having a porosity of less than or equal to 150 nanometers prior to the analysis stage and in particular, a porosity between 20 nanometers and 100 nanometers, a sensor for acquiring the electromagnetic signals transmitted by a solution, a circuit for processing said signals for calculating a signature for an analysed sample and a comparison circuit for comparing the signature so computed with a base of previously recorded signatures
